(19) United States
(12) Patent Application Publication
(10) Pub. No.: US 2014/0271663 A1
(43) Pub. Date: Sep. 18, 2014

(54) USE OF ANTI-EOTAXIN ANTIBODIES FOR TREATING INFLAMMATORY BOWEL DISEASE

(71) Applicant: IMMUNE PHARMACEUTICALS LTD., Herzlia (IL)

(72) Inventor: Daniel Teper, Herzlia Pituach (IL)

(73) Assignee: Immune Pharmaceuticals Ltd., Herzlia (IL)

(21) Appl. No.: 13/803,646

(22) Filed: Mar. 14, 2013

(51) Int. Cl. C07K 16/24

(52) U.S. Cl. CPC C07K 16/24
USPC 424/158.1

(57) ABSTRACT

The present invention provides use of one or more complementary determining regions (CDRs) of the CAT-212-213 VH and/or VL domains in non-native antibody framework regions, or, alternatively, the whole VH, VL, or CAT-212 antibody, in treating inflammatory diseases in a subject, such as inflammatory bowel disease.

### FIELD OF THE INVENTION

The present invention provides use of one or more complementary determining regions (CDRs) of the CAT-212-213 VH and/or VL domains in non-native antibody framework regions, or, alternatively, the whole VH, VL, or CAT-212 antibody, in treating inflammatory diseases in a subject, such as inflammatory bowel disease.

### BACKGROUND OF THE INVENTION

Human eotaxin is a member of the rapidly expanding group of the CC (Cys-Cys) subfamily of chemokines. This group of molecules is characterised by the presence of 4 conserved cysteines, the first 2 of which are adjacent and share a sequence identity between 20 and 75%. Members of this family include eotaxin-2, eotaxin-3, monocyte chemoattractant protein (MCP)-1, MCP-2, MCP-3, MCP-4, MCP-5, macrophage inflammatory protein (MIP)-1, MIP-13, TARC, LARC, 1309 and RANTES.

Eotaxin can be produced by a variety of normal cell types including epithelial cells, fibroblasts, endothelial cells, T-lymphocytes, monocytes and macrophages. Eotaxin expression can be induced from the different cell types by many pro-inflammatory mediators, such as tumour necrosis factor-alpha, interferon and interleukin-1.

Eotaxin-1 is a chemoattractant protein that binds to a specific receptor, CCR3, which is expressed predominantly on eosinophils and recruits eosinophils to tissues. On binding CCR3 on eosinophils, eotaxin causes intracellular calcium mobilisation, initiation of intracellular actin polymerisation, upregulation of integrin expression and the induction of oxygen radical production.

Eosinophils are proinflammatory leucocytes that constitute a small percentage of circulating blood cells. In the healthy state, most of these cells reside in the gastrointestinal tract within the lamina propria of the stomach and intestine.

Eosinophils secrete toxic inflammatory mediators that are stored in preformed vesicles and also synthesised de novo following cellular activation. The major proteins secreted by eosinophils are eosinophilic cationic protein, major basic protein, eosinophil protein X, eosinophil derived neuroendotoxin, and eosinophil peroxidase. These cause damage to tissues, insert pores into membranes of target cells, and increase smooth muscle reactivity by generating toxic oxygen radicals.

Eosinophils are believed play a role in inflammatory diseases of the gastrointestinal tract, such as inflammatory bowel disease (IBD)

The term inflammatory bowel disease (“IBD”) describes a group of chronic inflammatory disorders of unknown causes in which the intestine (bowel) becomes inflamed, often causing recurring cramps or diarrhea. IBD is generally divided into ulcerative colitis (UC) and Crohn's disease. The inflammatory process in these illnesses involves many inflammatory cells, such as lymphocytes, macrophages, mast cells, neutrophils, and eosinophils. The two most important roles that eosinophils play in IBD appear to be as proinflammatory and promotility agents thus producing effects such as diarrhea, inflammation, tissue destruction, formation of fibrosis and strictures and, as recently suggested, even repair.

In UC, the inflammatory response is confined to the mucosa and submucosa of the colon with clear demarcations. In Crohn's disease, the entire gastrointestinal tract can be involved and the inflammation can extend through the intestinal wall from mucosa to serosa. Areas of inflammation may be interspersed with relatively normal mucosa. In Crohn's disease, the predominant symptoms are diarrhea, abdominal pain and weight loss whereas in UC diarrhea is the main symptom, often accompanied by rectal bleeding. Both diseases are common in the industrialized world, with highest incidences in North America and Northern Europe. The peak age of onset for both diseases is between 15 and 30 years with a second minor peak between 55 and 80 years. Crohn's disease shows a higher incidence in females than in males.

Satisfactory treatment of IBD is an unmet medical need, as existing therapeutics have not been successful in curtailing the disease and preventing surgeries. Up to forty percent of all ulcerative colitis patients undergo surgery, which typically includes the removal of part of the large intestine or a full colostomy because of massive bleeding, chronic debilitating illness, performation of the colon, or risk of cancer. Such surgery is not curative for Crohn's disease, as 75% of all patients undergo at least one surgery in their lifetime, and up to 90% of these patients require additional surgeries. Consequently a therapeutic that can successfully treat inflammatory bowel disease will have the beneficial effects of improving a patient's quality of life, while potentially saving the healthcare system millions of dollars in costs associated with invasive surgical procedures.

### SUMMARY OF THE INVENTION

In one embodiment, the present invention provides methods for treating an inflammatory bowel disease in a subject, comprising administering a composition comprising a specific binding member that binds human eotaxin to said subject.

In one embodiment, the binding member comprises an antibody VH domain which comprises a VH CDR1, a VH CDR2 and a VH CDR3, wherein said VH CDR1, VH CDR2 and VH CDR3 consist of the amino acid sequences of SEQ ID NO. 5, SEQ ID NO. 6 and SEQ ID NO. 7, respectively.

In another embodiment, the antibody VH domain comprises SEQ ID NO. 2.

In another embodiment, the binding member comprises an antibody VL domain comprising a VL CDR1, a VL CDR2 and a VL CDR3.

In another embodiment, the VL CDR1 consists of the amino acid sequence of SEQ ID NO. 8.

In another embodiment, the VL CDR2 consists of the amino acid sequence of SEQ ID NO. 9.

In another embodiment, the VL CDR3 consists of the amino acid sequence of SEQ ID NO. 10.

In another embodiment, the antibody VL domain comprises SEQ ID NO. 4.

In one embodiment, the inflammatory bowel disease is ulcerative colitis.

In another embodiment, the inflammatory bowel disease is Crohn's Disease.

In another embodiment, the inflammatory bowel disease is Collagenous colitis, Lymphocytic colitis, Ischaemic colitis, Diversion colitis, Behçet's disease, or Indeterminate colitis.

### DETAILED DESCRIPTION OF THE PRESENT INVENTION

In one embodiment, the present invention provides methods for treating or preventing an inflammatory bowel disease in a subject, comprising administering a composition comprising a specific binding member that binds human eotaxin to said subject.

In another embodiment, the present invention provides methods for inhibiting or suppressing an inflammatory bowel disease in a subject, comprising administering a composition comprising a specific binding member that binds human eotaxin to said subject.

In another embodiment, the present invention provides methods for decreasing the incidence of an inflammatory bowel disease in a subject, comprising administering a composition comprising a specific binding member that binds human eotaxin to said subject.

In another embodiment, the present invention provides methods for inhibiting or neutralising eotaxin in a subject, comprising administering a composition comprising a specific binding member that binds human eotaxin to said subject.

In another embodiment, the present invention provides methods for competing with eotaxin activators for binding to eotaxin comprising administering a composition comprising a specific binding member that binds human eotaxin to said subject. In another embodiment, the present invention provides methods for competing with eotaxin receptors for binding sites to eotaxin comprising administering a composition comprising a specific binding member that binds human eotaxin to said subject.

In another embodiment, the present invention provides methods for blocking binding to eotaxin comprising administering a composition comprising a specific binding member that binds human eotaxin to said subject. In one embodiment, said binding member blocks binding of an eotaxin activator. In another embodiment, said binding member blocking binding of an eotaxin receptor.

In one embodiment, the present invention provides methods and uses for a composition comprising a specific binding member which binds human eotaxin, as described herein. In one embodiment, the composition includes at least one additional component, such as a pharmaceutically acceptable excipient.

In one embodiment, the present invention provides methods and uses for a specific binding member which binds human eotaxin. In one embodiment the binding member comprises the CAT-212 VH domain:

(SEQ ID NO. 2)
QVQLVQSGGG VVQPGRSLRL SCAASGFTFS SYGMHWVRQA
PGKGLEWVAV ISYDGSIKHY ADSVKGRFTI SRDNSKNTLY
LQMNSLRTDD TAVYYCAGDT DYGDIDPWGQ GTMVTVSS.

In one embodiment, the binding member comprises the CAT-212 VL domain:

(SEQ ID NO. 4)
DIQMTQSPSS VSASVGDRVT ITCRASQDIS SWLAWYQQKP
GKAPKLLIYA ASSLQSGVPS RFSGSGSGTD FTLTISSLQP
EDFATYYCQQ ASSFPSITFG QGTRLEIKR.

In another embodiment, the specific binding member that binds human eotaxin is a binding member that is known in the art. In one embodiment, the binding member is a human anti-eotaxin1 antibody known in the art.

Generally, a VH domain is paired with a VL domain to provide an antibody antigen binding site, although a VH domain alone may be used to bind antigen. In one preferred embodiment, the CAT-212 VH domain (SEQ ID NO. 2) is paired with the CAT-212 VL domain (SEQ ID NO. 4), so that an antibody antigen binding site is formed comprising both the CAT-212 VH and VL domains. In other embodiments, the CAT-212 VH is paired with a VL domain other than the CAT-212 VL. Light-chain promiscuity is well established in the art.

The specific binding member described herein which binds human eotaxin is described in further detail in U.S. Pat. Nos. 6,946,546; 7,323,311; 8,067,564; and US Patent Application Publication No: US 2012-0270265, which are incorporated herein by reference in their entirety, including methods for producing such binding members and related variants and the binding and other characteristics of the binding member.

In brief, it has been shown that a human single-chain fragment variable antibody that neutralizes human eotaxin1 (CAT-212) was produced using antibody phage display and converted to whole antibody IgG4 format (CAT-213). The length of the variable heavy chain complementarity-determining region 3 was reduced by one amino acid resulted in an increase in potency of >1000-fold compared with the parent anti-eotaxin1 antibody. The optimized antibody binds eotaxin1 with high affinity (80.4 pM) and specificity. CAT-213 and CAT-212 do not bind or neutralize a range of other human proteins including human monocyte chemoattractant protein-1, a structurally similar chemokine. CAT-213 neutralizes the ability of eotaxin1 to cause an increase in intracellular calcium signaling (with an IC(50) value of 2.86 nM), migration of CCR3-expressing L1.2 cells (with an IC(50) value of 0.48 nM), and inhibition of the eotaxin1-evoked shape change of human eosinophils in vitro (with an IC(50) of 0.71 nM). Local administration of CAT-213 to mice (1-100 microg kg(−1)) attenuates dermal eosinophilia induced by human eotaxin1, achieving >90% inhibition of eosinophil influx CAT-213 may therefore be of therapeutic value in inhibiting diseases in which eotaxin1 and eosinophils play a major role, for example, severe asthma.

In one embodiment, one or more CDRs may be taken from the CAT-212 VH or VL domain and incorporated into a suitable framework. CAT-212 VH comprises CDR 1 (SYGMH, SEQ ID NO: 5), CDR 2 (VISYDGSIKH YADSVKG, SEQ ID NO: 6) and CDR 3 (DTDYGDIDP, SEQ ID NO: 7). CAT-212 VL comprises CDR 1 (RASQDISSWLA, SEQ ID NO: 8), CDR 2 (AASSLQS, SEQ ID NO: 9) and CDR 3 (QQASSFPSIT, SEQ ID NO: 10).

Variants of the VH and VL domains and CDRs of which the sequences are set out herein and which can be employed in specific binding members for eotaxin can be obtained by means of methods of sequence alteration or mutation and screening. Such methods are also provided by the present invention.

Variable domain amino acid sequence variants of any of the VH and VL domains whose sequences are specifically disclosed herein may be employed in accordance with the present invention, as discussed. Particular variants may include one or more amino acid sequence alterations (addition, deletion, substitution and/or insertion of an amino acid residue), maybe less than about 20 alterations, less than about 15 alterations, less than about 10 alterations or less than about alterations, 4, 3, 2 or 1. Alterations may be made in one or more framework regions and/or one or more CDR's.

In addition to antibody sequences, a specific binding member according to the present invention may comprise other amino acids, e.g. forming a peptide or polypeptide, such as a folded domain, or to impart to the molecule another functional characteristic in addition to ability to bind antigen. Specific binding members of the invention may carry a detectable label, or may be conjugated to a toxin or enzyme (e.g. via a peptidyl bond or linker).

In further aspects, the invention provides an isolated nucleic acid which comprises a sequence encoding a specific binding member, VH or VL domains according to the present invention, and methods of preparing a specific binding member, a VH domain and/or a VL domain of the invention, which comprise expressing said nucleic acid under conditions to bring about production of said specific binding member, VH domain and/or VL domain, and recovering it.

The structure for carrying a CDR of the invention will generally be of an antibody heavy or light chain sequence or substantial portion thereof in which the CDR is located at a location corresponding to the CDR of naturally occurring VH and VL antibody variable domains encoded by rearranged immunoglobulin genes. The structures and locations of immunoglobulin variable domains may be determined by reference to Kabat, E. A. et al, Sequences of Proteins of Immunological Interest. 4th Edition. US Department of Health and Human Services. 1987, and updates thereof, now available on the Internet (http://immuno.bme.nwu.edul).

Preferably, a CDR amino acid sequence substantially as set out herein is carried as a CDR in a human variable domain or a substantial portion thereof. The VH CDR3 sequences substantially as set out herein represent preferred embodiments of the present invention and it is preferred that each of these is carried as a VH CDR3 in a human heavy chain variable domain or a substantial portion thereof.

Variable domains employed in the invention may be obtained from any germline or rearranged human variable domain, or may be a synthetic variable domain based on consensus sequences of known human variable domains. A CDR sequence of the invention (e.g. CDR3) may be introduced into a repertoire of variable domains lacking a CDR (e.g. CDR3), using recombinant DNA technology.

Techniques for doing this are known as such in the art and the skilled person will be able to use such techniques to provide specific binding members of the invention using routine methodology known in the art.

A substantial portion of an immunoglobulin variable domain will comprise at least the three CDR regions, together with their intervening framework regions. Preferably, the portion will also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain may be those not normally associated with naturally occurring-variable domain regions. For example, construction of specific binding members of the present invention made by recombinant DNA techniques may result in the introduction of NB or C-terminal residues encoded by linkers introduced to facilitate cloning or other manipulation steps. Other manipulation steps include the introduction of linkers to join variable domains of the invention to further protein sequences including immunoglobulin heavy. chains, other variable domains (for example in the production of diabodies) or protein labels as discussed in more details below.

Although in a preferred aspect of the invention specific binding members comprising a pair of VH and VL domains are preferred, single binding domains based on either VH or VL domain sequences form further aspects of the invention. It is known that single immunoglobulin domains, especially VH domains, are capable of binding target antigens in a specific manner.

Specific binding members of the present invention may further comprise antibody constant regions or parts thereof. For example, a VL domain may be attached at its C-terminal end to antibody light chain constant domains including human C.kappa. or C.lamda. chains, preferably C.lamda. chains. Similarly, a specific binding member based on a VH domain may be attached at its C-terminal end to all or part of an immunoglobulin heavy chain derived from any antibody isotype, e.g. IgG, IgA, IgE and IgM and any of the isotype sub-classes, particularly IgG1 and IgG4. IgG4 is preferred.

Specific binding members of the invention may be labelled with a detectable or functional label. Detectable labels include radiolabels such as .sup.131I or .sup.99Tc, which may be attached to antibodies of the invention using conventional chemistry known in the art of antibody imaging. Labels also include enzyme labels such as horseradish peroxidase. Labels further include chemical moieties such as biotin which may be detected via binding to a specific cognate detectable moiety, e.g. labelled avidin.

Specific binding members of the present invention are designed to be used in methods of diagnosis or treatment in human or animal subjects, preferably human. In one embodiment, a “subject” as used herein includes any mammalian subject, such as primate (human and non-human), mice, rats, other murine species, dogs, cats, horses, cattle, sheep and pigs, for example. In one embodiment, the subject is a companion animal. A companion animal refers to any non-human animal considered to be a pet, including but not limited to, dogs, cats, rabbits, monkeys, among others.

Specific binding members according to the invention may be used in a method of treatment or diagnosis of the human or animal body, such as a method of treatment (which may include prophylactic treatment) of a disease or disorder in a human patient which comprises administering to said patient an effective amount of a specific binding member of the invention.

Accordingly, further aspects of the invention provide methods of treatment comprising administration of a specific binding member as provided, pharmaceutical compositions comprising such a specific binding member, and use of such a specific binding member in the manufacture of a medicament for administration, for example in a method of making a medicament or pharmaceutical composition comprising formulating the specific binding member with a pharmaceutically acceptable excipient.

In one embodiment, the present invention provides methods for treating or preventing chronic relapsing inflammatory conditions in a subject, comprising administering a composition comprising a specific binding member that binds human eotaxin to said subject.

In another embodiment, the present invention provides methods for treating or preventing disorders related to inflammation in a subject, comprising administering a composition comprising a specific binding member that binds human eotaxin to said subject. In another embodiment, the present invention provides methods for treating or preventing an inflammatory disorder in a subject, comprising administering a composition comprising a specific binding member that binds human eotaxin to said subject.

In another embodiment, the present invention provides methods for treating or preventing a condition, disease, or disorder in a subject with high eosinophil and eotaxin-1 concentrations in their sputum, comprising administering a composition comprising a specific binding member that binds human eotaxin to said subject.

In another embodiment, the present invention provides methods for prevent or reduce eosinophil accumulation in the tissue of a subject, comprising administering a composition comprising a specific binding member that binds human eotaxin to said subject. In another embodiment, the present invention provides methods for preventing tissue injury and/or inflammation that results from eosinophil accumulation in the tissue of a subject.

In another embodiment, the present invention provides methods for treating or preventing auto-immune disorders in a subject, comprising administering a composition comprising a specific binding member that binds human eotaxin to said subject.

In one embodiment, an anti-eotaxin antibody may be used to treat subjects with inflammatory bowel disease (which in one embodiment, is ulcerative colitis or Crohn's disease) and eosinophilic colitis/enteritis/gastroenteritis/Shulman's syndrome and/or to suppress or inhibit symptoms of inflammatory bowel disease. In one embodiment, an anti-eotaxin antibody may be used to decrease the incidence of inflammatory bowel disease in a population, which in one embodiment, is a population susceptible to inflammatory bowel disease, whether by genetic predisposition, environmental factors, lifestyle habits, or a combination thereof.

In another embodiment, the inflammatory bowel disease is collagenous colitis, lymphocytic colitis, ischaemic colitis, diversion colitis, behçet's disease, or indeterminate colitis.

Eosinophils appear as a prominent cell-type in the lesions that characterise these diseases.

Vasculitis of several forms, especially idiopathic, Hugues-Stovin syndrome, Churg-Strauss syndrome, bronchocentric granulomatosis, eosinophilic pneumonitis (Loffler's syndrome), prolonged pulmonary eosinophilia, Omenn's syndrome, Wiskott-Aldrich syndrome, familial eosinophilia, and idiopathic hypereosinophilia may be treated with anti-eotaxin.

Eosinophilia of unknown cause can result complications such as pneumonitis, vasculitis, colitis, enteritis, gastroenteritis, Loffler's endocarditis and heart valve fibrosis and many syndromes affecting connective tissue. Eosinophilia can also be associated with malignant disease (especially lymphomas, leukaemias and gastrointestinal cancers), drug treatments (eg cytokine infusions) and chronic fatigue syndrome. Anti-eotaxin treatment may be employed in any of these diseases. Similarly, eosinophilia-myalgia syndrome, toxic-oil syndrome, diffuse fasciitis with eosinophilia (eosinophilic fasciitis) and eosinophilic myositis may be treated with anti-eotaxin.

The eosinophil attraction caused by parasites may be a harmful effect so intervention with anti-eotaxin in these conditions may provide benefit. The diseases involving eosinophil attraction by pathogens include protozoal infection, and metazoan infections such as helmith infestation and especially nematode infections (eg filariasis, hookworm, onchocerciasis, toxocariasis, ascariasis and trichinosis, angiostrongyliasis [eosinophilic meningitis]). Asymptomatic parasitic disease may be the cause of many of the idiopathic forms of eosinophil-mediated disease.

Anti-eotaxin treatment may have an effect on cells other than eosinophils, e.g. those expressing CCR-3 such as basophils.

Additional clinical indications in which an anti-eotaxin antibody may be used to provide therapeutic benefit include asthma, eczema (atopic dermatitis) and other atopic diseases such as rhinitis, conjunctivitis, food allergy, allergic colitis which are recognised as eosinophil-mediated diseases. Experimental evidence favours eosinophils as a cause of most cases of atopy so anti-eotaxin treatment is likely to be effective for all these diseases. There are other allergic conditions, such as allergic bronchopulmonary aspergillosis and tropical eosinophilia that feature high peripheral eosinophil counts and which may be subject to anti-eotaxin treatment.

According to this aspect and in one embodiment, the present invention provides methods for treating or preventing asthma in a subject, comprising administering a composition comprising a specific binding member that binds human eotaxin to said subject. In one embodiment, the asthma is severe asthma.

There is a clear need for improved treatment both for preventing asthma symptoms and to treat more severe symptoms once they have developed. Anti-eotaxin treatment may be given orally, by injection (for example, subcutaneously or in emergencies, intravenously), by inhalation (to optimise the profile of beneficial effects compared with any unwanted effects) or by alternative routes of administration. The route of administration may be determined by the physicochemical characteristics of the treatment, by special considerations for the disease, to optimise efficacy or to minimise side-effects.

In addition, the present invention provides methods for treating or preventing allergic diseases in a subject, comprising administering a composition comprising a specific binding member that binds human eotaxin to said subject. In one embodiment, said allergic disease is conjunctivitis. In another embodiment, said allergic disease is rhinitis.

Skin conditions may best be treated with topical treatment with anti-eotaxin. Diseased skin often has increased absorptive capacity, compared with healthy skin, so topical treatment may well provide the best route for therapy, where it is needed, without unwanted effects elsewhere in the body. If the skin condition covers much of the body, or if the disease is severe (maybe affecting other organs as well as the skin) then administration by injection or by other efficient means may be more appropriate that the topical route. Local injection may be appropriate under certain circumstances (see the previous paragraph).

According to this aspect and in one embodiment, the present invention provides methods for treating or preventing skin diseases or dermatological disorders in a subject, comprising administering a composition comprising a specific binding member that binds human eotaxin to said subject. In one embodiment, the skin disease is an inflammatory skin disease. In one embodiment, the inflammatory skin disease is atoptic dermatitis. In one embodiment said dermatological disorder is bullous pemphigold which is a rare autoimmune skin disease characterized by activation of inflammatory cells, resulting in skin lesions in patients.

In another embodiment, the present invention provides methods for inhibiting angiogenesis in a subject, comprising administering a composition comprising a specific binding member that binds human eotaxin to said subject.

In another embodiment, the present invention provides methods for treating or preventing a cell proliferative disorder in a subject, comprising administering a composition comprising a specific binding member that binds human eotaxin to said subject. In another embodiment, the present invention provides methods for treating or preventing cancer in a subject, comprising administering a composition comprising a specific binding member that binds human eotaxin to said subject. In another embodiment, the present invention provides methods for treating or preventing tumor development in a subject, comprising administering a composition comprising a specific binding member that binds human eotaxin to said subject. In another embodiment, the present invention provides methods for treating or preventing growth of a precancerous lesion in a subject, comprising administering a composition comprising a specific binding member that binds human eotaxin to said subject.

In one embodiment, said cancer or tumor is a breast cancer or tumor. In another embodiment, said cancer or tumor is a lung, colon, colorectal, stomach, gastric intestinal, prostate, brain, liver, kidney, bladder, skin, pancreas, spleen, thymus, testis, ovary, cervix, or uterus cancer or tumor. In one embodiment, said brain cancer is a glioblastoma.

In another embodiment, the present invention provides methods for treating or preventing a chronic eye disease in a subject, comprising administering a composition comprising a specific binding member that binds human eotaxin to said subject. In one embodiment, the chronic eye disease is vernal keratoconjunctivitis (VKC). In another embodiment, the chronic eye disease is atopic keratoconjunctivitis (AKC).

In another embodiment, the present invention provides methods for treating or preventing a gastroenterology, oncology, dermatology, ophthalmology, respiratory, dermatology, or neurology-related disorder in a subject, comprising administering a composition comprising a specific binding member that binds human eotaxin to said subject. In another embodiment, the present invention provides methods for treating or preventing age-related cognate decline (“ACD”) in a subject, comprising administering a composition comprising a specific binding member that binds human eotaxin to said subject.

It is envisaged that anti-eotaxin treatment will not be restricted to use in the clinic. Patients may self-administer the treatment and daily administration may be preferred over complex dosing schedules.

Combination treatments may be used to provide significant synergistic effects, particularly the combination of an anti-eotaxin specific binding member with one or more anti-interleukin-5 (IL-5) drugs. A specific binding member according to the present invention may be provided in combination or addition to one or more corticosteroids, particularly one or more systemic corticosteroids. Combination treatment with one or more other anti-asthma/anti-allergy agents, especially other Apreventers@ such as cromoglycate, leukotriene (receptor) antagonists, xanthines and long-acting bronchodilators may be employed for asthma treatment. Similar considerations of combinations apply to the use of anti-eotaxin treatment for skin and other atopic conditions.

All forms of psoriasis, urticaria (including acute urticaria, chronic recurrent urticaria, delayed pressure urticaria, cold urticaria, dermographic urticaria), prurigo nodularis, papular erythematous eruptions, pemphigoid, porphyria cutanea tarda, persistent light reaction, Wells' syndrome, eosinophilic cellulitis, drug eruptions, vasculitis (skin manifestation), purpura and other skin conditions may be treated with anti-eotaxin in accordance with the present invention. These conditions can cover a large proportion of the body, may involve organs other than the skin or may not cause the skin to have increased permeability. Even if effective applied topically, at the site of action, the preferred route may be systemic (through the body) for the same considerations as suggested for atopic indications. Severe skin disease with associated systemic manifestations is a good example of a situation in which systemic treatment may be preferred to topical treatment or local injection.

In accordance with the present invention, compositions provided may be administered to individuals. Administration is preferably in a “therapeutically effective amount”, this being sufficient to show benefit to a patient. Such benefit may be at least amelioration of at least one symptom. The actual amount administered, and rate and time-course of administration, will depend on the nature and severity of what is being treated. Prescription of treatment, eg decisions on dosage etc, is within the responsibility of general practitioners and other medical doctors. Appropriate doses of antibody are well known in the art; see Ledermann J. A. et al. (1991) Int J. Cancer 47: 659-664; Bagshawe K. D. et al. (1991) Antibody, Immunoconjugates and Radiopharmaceuticals 4: 915-922.

The precise dose will depend upon a number of factors, including whether the antibody is for diagnosis or for treatment, the size and location of the area to be treated, the precise nature of the antibody (e.g. whole antibody, fragment or diabody), and the nature of any detectable label or other molecule attached to the antibody. A typical antibody dose will be in the range 0.5 mg to 100 g for systemic applications, and 10 .mu.g to 1 mg for local applications. Typically, the antibody will be a whole antibody, preferably the IgG4 isotype. This is a dose for a single treatment of an adult patient, which may be proportionally adjusted for children and infants, and also adjusted for other antibody formats in proportion to molecular weight. Treatments may be repeated at daily, twice-weekly, weekly or monthly intervals, at the discretion of the physician.

Specific binding members of the present invention will usually be administered in the form of a pharmaceutical composition, which may comprise at least one component in addition to the specific binding member.

Thus pharmaceutical compositions according to the present invention, and for use in accordance with the present invention, may comprise, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

A composition may be administered alone or in combination with other treatments, either simultaneously or sequentially dependent upon the condition to be treated. Other treatments may include the administration of suitable doses of pain relief drugs such as non-steroidal anti-inflammatory drugs (e.g. asprin, paracetamol, ibuprofen or ketoprofen) or opiates such as morphine, or anti-emetics.

The present invention provides a method comprising causing or allowing binding of a specific binding member as provided herein to eotaxin. As noted, such binding may take place in vivo, e.g. following administration of a specific binding member, or nucleic acid encoding a specific binding member, or it may take place in vitro, for example in ELISA, Western blotting, immunocytochemistry, immuno-precipitation or affinity chromatography.

The amount of binding of specific binding member to eotaxin may be determined. Quantitation may be related to the amount of the antigen in a test sample, which may be of diagnostic interest, which may be of diagnostic interest.

The reactivities of antibodies on a sample may be determined by any appropriate means. Radioimmunoassay (RIA) is one possibility. Radioactive labelled antigen is mixed with unlabelled antigen (the test sample) and allowed to bind to the antibody. Bound antigen is physically separated from unbound antigen and the amount of radioactive antigen bound to the antibody determined. The more antigen there is in the test sample the less radioactive antigen will bind to the antibody. A competitive binding assay may also be used with non-radioactive antigen, using antigen or an analogue linked to a reporter molecule. The reporter molecule may be a fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotih/streptavidin and alkaline phosphatase detection systems may be employed.

The signals generated by individual antibody-reporter conjugates may be used to derive quantifiable absolute or relative data of the relevant antibody binding in samples (normal and test).

The present invention also provides the use of a specific binding member as above for measuring antigen levels in a competition assay, that is to say a method of measuring the level of antigen in a sample by employing a specific binding member as provided by the present invention in a competition assay. This may be where the physical separation of bound from unbound antigen is not required. Linking a reporter molecule to the specific binding member so that a physical or optical change occurs on binding is one possibility. The reporter molecule may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule.

The present invention also provides for measuring levels of antigen directly, by employing a specific binding member according to the invention for example in a biosensor system.

The mode of determining binding is not a feature of the present invention and those skilled in the art are able to choose a suitable mode according to their preference and general knowledge.

The present invention further provides an isolated nucleic acid encoding a specific binding member of the present invention. In one embodiment, a nucleic acid is a DNA sequence. In another embodiment, a nucleic acid is an RNA sequence. In one embodiment, the present invention provides a nucleic acid which codes for a CDR, VH domain, VL domain, or a combination thereof of the invention as defined herein. In one embodiment, the nucleic acid encoding the VH domain is:

(SEQ ID NO: 1)
CAGGTGCAGCTGGTGCAATCTGGGGGAGGCGTGGTCCAGCCTGGGAGGTC
CCTGAGACTCTCCTGTGCAGCCTCTGGATTCACCTTCAGTAGCTATGGCA
TGCACTGGGTCCGCCAGGCTCCAGGCAAGGGGCTGGAGTGGGTGGCAGTT
ATATCATATGATGGAAGCATTAAACATTATGCAGACTCCGTGAAGGGCCG
ATTCACCATCTCCAGAGACAATTCCAAGAACACGCTGTATCTGCAAATGA
ACAGCCTGAGAACTGACGACACGGCTGTATATTACTGTGCGGGAGATACG
GACTACGGGGACATCGACCCGTGGGGTCAGGGCACCATGGTGACGGTCTC
GAGT.

In one embodiment, the nucleic acid encoding the VH domain is:

(SEQ ID NO: 3)
ACATCCAGATGACCCAGTCTCCATCTTCCGTGTCTGCATCTGTAGGAGAC
AGAGTCACCATCACTTGTCGGGCGAGTCAGGATATTAGCAGCTGGTTAGC
CTGGTATCAGCAGAAACCTGGGAAAGCCCCTAAGCTCCTGATCTATGCTG
CATCCAGTTTGCAAAGTGGGGTCCCATCAAGGTTCAGCGGCAGTGGATCT
GGGACAGATTTCACTCTCACCATCAGCAGCCTGCAGCCTGAAGATTTTGC
AACTTACTATTGTCAGCAGGCTAGCAGTTTCCCCTCGATCACCTTCGGCC
AAGGGACACGACTGGAGATTAAACGT.

The present invention also provides constructs in the form of plasmids, vectors, transcription or expression cassettes which comprise at least one polynucleotide as above.

The present invention also provides a recombinant host cell which comprises one or more constructs as above. A nucleic acid encoding any CDR, VH or VL domain, or specific binding member as provided itself forms an aspect of the present invention, as does a method of production of the encoded product, which method comprises expression from encoding nucleic acid therefor. Expression may conveniently be achieved by culturing under appropriate conditions recombinant host cells containing the nucleic acid. Following production by expression a VH or VL domain, or specific binding member may be isolated and/or purified using any suitable technique, then used as appropriate.

Specific binding members, VH and/or VL domains, and encoding nucleic acid molecules and vectors according to the present invention may be provided isolated and/or purified, e.g. from their natural environment, in substantially pure or homogeneous form, or, in the case of nucleic acid, free or substantially free of nucleic acid or genes origin other than the sequence encoding a polypeptide with the required function. Nucleic acid according to the present invention may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of antibodies and antibody fragments in prokaryotic cells such as E. coli is well established in the art. For a review, see for example Pluckthun, A. Bio/Technology 9: 545-551 (1991). Expression in eukaryotic cells in culture is also available to those skilled in the art as an option for production of a specific binding member, see for recent reviews, for example Ref, M. E. (1993) Curr. Opinion Biotech. 4: 573-576; Trill J. J. et al. (1995) Curr. Opinion Biotech 6: 553-560.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992. The disclosures of Sambrook et al. and Ausubel et al. are incorporated herein by reference.

Thus, a further aspect of the present invention provides a host cell containing nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage.

The introduction may be followed by causing or allowing expression from the nucleic acid, e.g. by culturing host cells under conditions for expression of the gene.

In one embodiment, the nucleic acid of the invention is integrated into the genome (e.g. chromosome) of the host cell. Integration may be promoted by inclusion of sequences which promote recombination with the genome, in accordance with standard techniques.

The present invention also provides a method which comprises using a construct as stated above in an expression system in order to express a specific binding member or polypeptide as above.

In one embodiment, IBD or colitis is assessed by endoscopy. In one embodiment, IBD or colitis is assessed by analysis of pro-inflammatory chemokines and cytokines, which in one embodiment, are KC, IL-1beta, TNFalpha, IL-6, IFN-gamma, IL-10, or a combination thereof. In another embodiment, IBD or colitis is assessed using measurement of feces osmolarity. In another embodiment, IBD or colitis is assessed by measuring epithelium resistance using Electric Cell-substrate Impedance Sensing (ECIS). These techniques are known in the art.

In another embodiment, the present invention provides methods for diagnosing an eosinophil-related disease, condition or disorder in a subject, comprising administering a composition comprising a specific binding member that binds human eotaxin to said subject. In one embodiment, the binding member for use in diagnosing is labeled. In one embodiment, the binding member for use in diagnosing is a probe. In another embodiment, the method further comprises the step of detecting the label to quantitatively determine the level of eotaxin in a region of interest in said subject. In one embodiment, the region of interest is the digestive tract. In another embodiment, the region of interest is the intestine. In another embodiment, the region of interest is the stomach. In another embodiment, the region of interest is the colon. In another embodiment, the diagnostic method further comprises the step of treating said eosinophil-related disease, condition or disorder if the subject is diagnosed with an eosinophil-related disease, condition or disorder. In another embodiment, the specific binding member is used for both diagnosing and treating said eosinophil-related disease, condition or disorder, and in one embodiment, said diagnosing and treating is achieved simultaneously. In one embodiment, said eosinophil-related disease, condition or disorder is inflammatory bowel disease. In one embodiment, in vivo imaging is used to detect a labeled specific binding member. In another embodiment, the method of diagnosing an eosinophil-related disease, condition or disorder in a subject comprises the step of isolating a sample of tissue from said subject and contacting said sample with said specific binding member ex vivo. In one embodiment, a sample, such as a biopsy, in one embodiment, are taken from a tissue of healthy subjects in order to establish a baseline value for eotaxin levels. In one embodiment, a sample taken from a subject is compared to the baseline value, and, if it exceeds the baseline value by a predetermined amount (in percent), then the subject is diagnosed with an eosinophil-related disease.

In another embodiment, a binding member of the present invention is administered to a subject as a single inoculation. In another embodiment, the binding member is administered twice. In another embodiment, the binding member is administered three times. In another embodiment, the binding member is administered four times. In another embodiment, the binding member is administered at least four times. In another embodiment, the binding member is administered more than four times. In the case where there are multiple administrations of the binding member, in one embodiment, the binding member is administered at separate sites, while in another embodiment, the binding member is administered each time at the same site. In another embodiment, the binding member is administered at 1 week intervals. In another embodiment, the binding member is administered at 2 week intervals. In another embodiment, the binding member is administered at 3 week intervals. In another embodiment, the binding member is administered at 4 week intervals. In another embodiment, the binding member is administered at 1 month intervals.

In one embodiment, methods of the present invention involve treating a condition, disease or disorder. In one embodiment, “treating” refers to a therapeutic treatment. In another embodiment, methods of the present invention involve preventing a disease or disorder, which in one embodiment, refers to prophylactic or preventative measures, wherein the object is to prevent or lessen the targeted pathologic condition or disorder as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, the compositions and methods of the present invention are effective in lowering IBD acquisition rates, the duration of IBD symptoms, the frequency of IBD symptoms, or a combination thereof.

### TERMINOLOGY

### Specific Binding Member

This describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organisation of the other member of the pair of molecules. Thus the members of the pair have the property of binding specifically to each other. Examples of types of specific binding pairs are antigen-antibody, biotin-avidin, hormone-hormone receptor, receptor-ligand, enzyme-substrate. This application is concerned with antigen-antibody type reactions.

Antibody

This describes an immunoglobulin whether natural or partly or wholly synthetically produced. The term also covers any polypeptide or protein having a binding domain which is, or is substantially homologous to, an antibody binding domain. Examples of antibodies are the immunoglobulin isotypes and their isotypic subclasses; fragments which comprise an antigen binding domain such as Fab, scFv, Fv, dAb, Fd; and diabodies.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin See, for instance, EP-A-184187, GB 2188638A or EP-A-239400. A hybridoma or other cell producing an antibody may be subject to genetic mutation or other changes, which may or may not alter the binding specificity of antibodies produced.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P. Holliger et al, Proc. Natl. Acad. Sci. USA 90 6444-6448, 1993). Fv, scFv or diabody molecules may be stabilised by the incorporation of disulphide bridges linking the VH and VL domains (Y. Reiter et al, Nature Biotech, 14, 1239-1245, 1996). Minibodies comprising a scFv joined to a CH3 domain may also be made (S. Hu et al, Cancer Res., 56, 3055-3061, 1996).

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804).

Where bispecific antibodies are to be used, these may be conventional bispecific antibodies, which can be manufactured in a variety of ways (Holliger, P. and Winter G. Current Opinion Biotechnol. 4, 446-449 (1993)), e.g. prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (J. B. B. Ridgeway et al, Protein Eng., 9, 616-621, 1996).

Antigen Binding Domain

This describes the part of an antibody which comprises the area which specifically binds to and is complementary to part or all of an antigen. Where an antigen is large, an antibody may only bind to a particular part of the antigen, which part is termed an epitope. An antigen binding domain may be provided by one or more antibody variable domains (e.g. a so-called Fd antibody fragment consisting of a VH domain). Preferably, an antigen binding domain comprises an antibody light chain variable region (VL) and an antibody heavy chain variable region (VH).

Specific

This may be used to refer to the situation in which one member of a specific binding pair will not show any significant binding to molecules other than its specific binding partner(s). The term is also applicable where e.g. an antigen binding domain is specific for a particular epitope which is carried by a number of antigens, in which case the specific binding member carrying the antigen binding domain will be able to bind to the various antigens carrying the epitope.

Isolated

This refers to the state in which specific binding members of the invention, or nucleic acid encoding such binding members, will be in accordance with the present invention. Members and nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practised in vitro or in vivo.

Members and nucleic acid may be formulated with diluents or adjuvants and still for practical purposes be isolated—for example the members will normally be mixed with gelatin or other carriers if used to coat microtitre plates for use in immunoassays, or will be mixed with pharmaceutically acceptable carriers or diluents when used in diagnosis or therapy. Specific binding members may be glycosylated, either naturally or by systems of heterologous eukaryotic cells (e.g. CHO or NSO (ECACC 85110503) cells, or they may be (for example if produced by expression in a prokaryotic cell) unglycosylated.

By “substantially as set out” it is meant that the relevant CDR or VH or VL domain of the invention will be either identical or highly similar to the specified regions of which the sequence is set out herein. By “highly similar” it is contemplated that from 1 to 5, preferably from 1 to 4 such as 1 to 3 or 1 or 2, or 3 or 4, substitutions may be made in the CDR and/or VH or VL domain.

In some embodiments, any of the binding members of and for use in the methods of the present invention will comprise the VH domain(s), VL domain(s), or specific sequence(s), of the present invention, or a combination thereof, as described herein, in any form or embodiment as described herein. In some embodiments, any of the binding members of and for use in the methods will consist of the VH domain(s), VL domain(s), or specific sequence(s) of the present invention, or a combination thereof, in any form or embodiment as described herein. In some embodiments, the binding members of this invention will consist essentially of the VH domain(s), VL domain(s), or specific sequence(s) of the present invention, or a combination thereof, in any form or embodiment as described herein. In some embodiments, the term “comprise” or “comprising” refers to the inclusion of other active ingredients, including other binding members or other agents meant to boost the efficacy or decrease the side effects of the binding member of the present invention. In some embodiments, the term “consisting essentially of” refers to a binding member, which has the specific VH domain(s), VL domain(s), or specific sequence(s), of the present invention, or a combination thereof. However, other elements may be included that are not involved directly in the utility of the VH domain(s), VL domain(s), or specific sequence(s), of the present invention. In some embodiments, the term “consisting of” refers to a binding member having the particularly described VH domain(s), VL domain(s), or specific sequence(s), of the present invention, or combination thereof in any form or embodiment as described herein.

In some embodiments, any of methods of the present invention will comprise the step of administering the VH domain(s), VL domain(s), or specific sequence(s), of the present invention, or a combination thereof, as described herein, in any form or embodiment as described herein. In some embodiments, any of the methods of the present invention will consist of administering the VH domain(s), VL domain(s), or specific sequence(s) of the present invention, or a combination thereof, in any form or embodiment as described herein. In some embodiments, the methods of the present invention will consist essentially of administering the VH domain(s), VL domain(s), or specific sequence(s) of the present invention, or a combination thereof, in any form or embodiment as described herein. In some embodiments, the term “comprise” or “comprising” refers to the inclusion of other active steps, including administering other binding members or other agents meant to boost the efficacy or decrease the side effects of the binding member of the present invention. In some embodiments, the term “consisting essentially of” refers to a method, which has mainly the specific steps described in the present invention. However, other steps may be included that are not involved directly in the method of administering the VH domain(s), VL domain(s), or specific sequence(s), of the present invention. In some embodiments, the term “consisting of” refers to a method having the particularly described steps of administering the VH domain(s), VL domain(s), or specific sequence(s), of the present invention, or combination thereof in any form or embodiment as described herein.

It is to be understood that the binding members of and for use in the present invention may be homologous to the binding members described herein, as long as they retain the anti-eotaxin binding function demonstrated by the binding members described herein. According to this aspect and in one embodiment, the binding members of and for use in the present invention are, in one embodiment, 70% homologous, in another embodiment, 80% homologous, in another embodiment, 85% homologous, in another embodiment, 90% homologous, in another embodiment, 95% homologous, and, in another embodiment, 98% to SEQ ID NOs: 2 and 4-10. In one embodiment, such homologous binding members may be useful in suppressing, inhibiting, preventing, or treating, one or more of the conditions, diseases or disorders in which eotaxin plays a role, as described herein.

In another embodiment, “homology” refers to identity to a sequence selected from SEQ ID NOs: 2, 4-10 of greater than 70%. In another embodiment, “homology” refers to identity to a sequence selected from SEQ ID NOs: 2, 4-10 of greater than 72%. In another embodiment, “homology” refers to identity to one of SEQ ID NOs: 2, 4-10 of greater than 75%. In another embodiment, “homology” refers to identity to a sequence selected from SEQ ID NOs: 2, 4-10 of greater than 78%. In another embodiment, “homology” refers to identity to one of SEQ ID NOs: 2, 4-10 of greater than 80%. In another embodiment, “homology” refers to identity to one of SEQ ID NOs: 2, 4-10 of greater than 82%. In another embodiment, “homology” refers to identity to a sequence selected from SEQ ID NOs: 2, 4-10 of greater than 83%. In another embodiment, “homology” refers to identity to one of SEQ ID NOs: 2, 4-10 of greater than 85%. In another embodiment, “homology” refers to identity to one of SEQ ID NOs: 2, 4-10 of greater than 87%. In another embodiment, “homology” refers to identity to a sequence selected from SEQ ID NOs: 2, 4-10 of greater than 88%. In another embodiment, “homology” refers to identity to one of SEQ ID NOs: 2, 4-10 of greater than 90%. In another embodiment, “homology” refers to identity to one of SEQ ID NOs: 2, 4-10 of greater than 92%. In another embodiment, “homology” refers to identity to a sequence selected from SEQ ID NOs: 2, 4-10 of greater than 93%. In another embodiment, “homology” refers to identity to one of SEQ ID NOs: 2, 4-10 of greater than 95%. In another embodiment, “homology” refers to identity to a sequence selected from SEQ ID NOs: 2, 4-10 of greater than 96%. In another embodiment, “homology” refers to identity to one of SEQ ID NOs: 2, 4-10 of greater than 97%. In another embodiment, “homology” refers to identity to one of SEQ ID NOs: 2, 4-10 of greater than 98%. In another embodiment, “homology” refers to identity to one of SEQ ID NOs: 2, 4-10 of greater than 99%.

In one embodiment, the terms “homology,” “homologous,” etc, when in reference to any protein or peptide, refer, in one embodiment, to a percentage of AA residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

Homology is, in another embodiment, determined by computer algorithm for sequence alignment, by methods well described in the art. For example, computer algorithm analysis of nucleic acid sequence homology can include the utilization of any number of software packages available, such as, for example, the BLAST, DOMAIN, BEAUTY (BLAST Enhanced Alignment Utility), GENPEPT and TREMBL packages.

In another embodiment, the present invention provides a kit comprising a vaccine utilized in performing a method of the present invention. In another embodiment, the present invention provides a kit comprising a vaccine of the present invention.

In one embodiment, the specific binding member of the present invention is administered to a subject. In one embodiment, “administering,” refers to directly introducing into a subject by injection or other means a composition of the present invention. In another embodiment, “administering” refers to contacting a cell of the subject's immune system with a binding member.

It is to be understood that any of the methods as described herein also describes various uses of the binding members described herein, such as, for example, a use of a specific binding member that binds human eotaxin in a method for treating or preventing an inflammatory bowel disease. Similarly, it is to be understood that any of the methods as described herein also describes the use of the binding members described herein in the preparation of a composition for treating an inflammatory bowel disease. For example, the present invention describes the use of a specific binding member that binds human eotaxin in the preparation of a composition for treating an inflammatory bowel disease.

Aspects and embodiments of the present invention will now be illustrated by way of example with reference to the following experimentation.

### Example 1

### Anti Eotaxin-1 Antibody is Therapeutic in Mice with DSS-Induced Colitis

Materials and Methods

All studies were performed in accordance with the Institutional Animal Care and Use Committee. Male Balb/C mice were used for this study.

Colitis was induced in all mice by providing drinking water to which 3.5% dextran sodium sulfate (DSS; molecular weight 42 kDa; ICN Biochemicals, Aurora, Ohio) had been added (day 1). Mice were treated i.p. with either isotype control antibody (mIgG2a (R&D #54447) or anti-Eotaxin-1 (mab 420, R&D, 4 mg/kg; 100 μg/animal) on days 0 (i.e., 24 h prior to DSS induction) and 4. Mice were sacrificed on day 7 and evaluated for (1) Disease activity index (including Body weight, Diarrhea, and Blood in stool); (2) Colon length and weight after resection (a marker of tissue edema); (3) H&E stain from colon; and (4) Tissue myeloperoxidase (MPO) activity.

Determination of Disease Activity Index (DAI)

In all animals, weight, stool blood, presence of gross blood and stool consistency were determined daily. Disease activity index (DAI) was determined by combining scores of a) weight loss b) stool consistency and c) bleeding (divided by 3). Each score was determined as follows, change in weight (0: <1%, 1: 1-5%, 2: 5-10%, 4: >15%), stool blood (0: negative, 2: positive) or gross bleeding (4), and stool consistency (0: normal, 2: loose stools, 4: diarrhea). Bodyweight loss was calculated as the percent difference between the original bodyweight and the actual bodyweight on any particular day. Typically in DSS colitis animals will lose 10-15% body weight over the course of 10 days. The appearance of diarrhea is defined as mucus/fecal material adherent to anal fur. The presence or absence of diarrhea was scored as either 1 or 0, respectively, and the cumulative score for diarrhea was calculated by adding the score for each day and dividing by the number of days of exposure. Rectal bleeding was defined as diarrhea containing visible blood/mucus or gross rectal bleeding and scored as described for diarrhea.

Histological Score Assessment of Colitis

H&E-stained colonic sections were coded for blind microscopic assessment of inflammation (i.e., DSS-induced colitis). Histological scoring was based on 3 parameters. Severity of inflammation was scored as follows: 0, rare inflammatory cells in the lamina propria; 1, increased numbers of granulocytes in the lamina propria; 2, confluence of inflammatory cells extending into the submucosa; 3, transmural extension of the inflammatory infiltrate. Crypt damage was scored as follows: 0, intact crypts; 1, loss of the basal one-third; 2, loss of the basal two-thirds; 3, entire crypt loss; 4, change of epithelial surface with erosion; 5, confluent erosion. Ulceration was scored as follows: 0, absence of ulcer; 1, 1 or 2 foci of ulcerations; 2, 3 or 4 foci of ulcerations; 3, confluent or extensive ulceration. Values were added to give a maximal histological score of 11.

Myeloperoxidase (MPO) Assay

Colonic tissue samples were homogenized in ice-cold potassium phosphate buffer (50 mM K2HPO4 and 50 mM KH2PO4, pH 6.0) containing 0.5% hexadecyltrimethylammonium bromide (Sigma). The homogenates were then sonicated, freeze-thawed three times, and centrifuged at 17,500 rcf for 15 min Supernatants (20 μl) or MPO standard were added to 1 mg/mL o-dianisidine hydrochloride (Sigma) and 0.0005% H2O2, and the change in absorbance at 450 nm was measured. One unit of MPO activity was defined as the amount that degraded 1 μmol peroxidase per minute. The results were expressed as relative MPO activity compared to water-treated mice (normalized to 1).

Results

Treatment of mice with anti-Eotaxin1 antibody prevented colitis development in the DSS colitis mouse model. Treatment of DSS colitis mice with anti-Eotaxin1 antibody improved the disease activity index in mice that received anti-Eotaxin1 antibody, and there was a minor decrease in body weight loss. Bleeding and diarrhea were ameliorated in DSS-treated mice who received anti-Eotaxin1 antibody. Finally, the ration of colon weight to length was lower in anti-eotaxin1-treated mice with DSS-induced colitis.

### Example 2

### Phase I Human Clinical Trials of CAT-212 for Ulcerative Colitis

A Phase I open-label, multicenter clinical trial to evaluate the safety and pharmacokinetics of CAT-212 in seventeen patients with ulcerative colitis (UC) is conducted. The diagnosis of total ulcerative colitis is made on the basis of x-ray and colonoscopic findings. The median age is 59 years (range 29-79). Patients stop treatment for ulcerative colitis (e.g., salazosulfapyridine, Predonine) 1 week prior to the trial. All patients receive a single dose of 3 mg/kg of CAT-212 intravenously over 90 minutes and are then followed for toxicity, pharmacokinetics, circulating T cell activation and clinical outcome. All infusions are completed with only mild adverse events. Seven patients have mild, reversible rashes or pruritis. Plasma levels of antibody persist from one to four months. There are no significant increase in activated peripheral T cells and no evidence of clinical autoimmunity beyond the mild rash.

In several patients, macroscopic mucus and blood in the stool and abdominal pain are alleviated on the day after the administration and completely disappear in the 2nd post-treatment week. Pulse rate and body temperature also begin to return to normal shortly after treatment and are completely normalized one week after the final administration.

The results of this study indicate that anti-eotaxin1 treatment is well tolerated with clear evidence of immunologic and therapeutic activity.

What is claimed is:

1. A method for treating an inflammatory bowel disease in a subject, comprising administering a composition comprising a specific binding member that binds human eotaxin to said subject.

2. The method of claim 1, wherein said binding member comprises an antibody VH domain which comprises a VH CDR1, a VH CDR2 and a VH CDR3, wherein said VH CDR1, VH CDR2 and VH CDR3 consist of the amino acid sequences of SEQ ID NO. 5, SEQ ID NO. 6 and SEQ ID NO. 7, respectively.

3. The method of claim 2, wherein said antibody VH domain comprises SEQ ID NO. 2.

4. The method of claim 1, wherein said binding member comprises an antibody VL domain comprising a VL CDR1, a VL CDR2 and a VL CDR3.

5. The method of claim 4, wherein said VL CDR1 consists of the amino acid sequence of SEQ ID NO. 8.

6. The method of claim 4, wherein said VL CDR2 consists of the amino acid sequence of SEQ ID NO. 9.

7. The method of claim 4, wherein said VL CDR3 consists of the amino acid sequence of SEQ ID NO. 10.

8. The method of claim 4, wherein said antibody VL domain comprises SEQ ID NO. 4.

9. The method of claim 1, wherein said inflammatory bowel disease is ulcerative colitis.

10. The method of claim 1, wherein said inflammatory bowel disease is Crohn's Disease.

11. The method of claim 1, wherein said inflammatory bowel disease is Collagenous colitis, Lymphocytic colitis, Ischaemic colitis, Diversion colitis, Behçet's disease, or Indeterminate colitis.


SEQUENCE LISTING (10 sequences)

SEQ ID NO: 1 - DNA, 354 bp, Homo sapiens
caggtgcagctggtgcaatctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagcattaaacattatgcagactccgtgaagggccgattcaccatctccagagacaattccaagaacacgctgtatctgcaaatgaacagcctgagaactgacgacacggctgtatattactgtgcgggagatacggactacggggacatcgacccgtggggtcagggcaccatggtgacggtctcgagt

SEQ ID NO: 2 - protein, 118 aa, Homo sapiens
QVQLVQSGGGVVQPGRSLRLSCAASGFTFSSYGMHWVRQAPGKGLEWVAVISYDGSIKHYADSVKGRFTISRDNSKNTLYLQMNSLRTDDTAVYYCAGDTDYGDIDPWGQGTMVTVSS

SEQ ID NO: 3 - DNA, 326 bp, Homo sapiens
acatccagatgacccagtctccatcttccgtgtctgcatctgtaggagacagagtcaccatcacttgtcgggcgagtcaggatattagcagctggttagcctggtatcagcagaaacctgggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcagcaggctagcagtttcccctcgatcaccttcggccaagggacacgactggagattaaacgt

SEQ ID NO: 4 - protein, 109 aa, Homo sapiens
DIQMTQSPSSVSASVGDRVTITCRASQDISSWLAWYQQKPGKAPKLLIYAASSLQSGVPSRFSGSGSGTDFTLTISSLQPEDFATYYCQQASSFPSITFGQGTRLEIKR

SEQ ID NO: 5 - protein, 5 aa, Homo sapiens
SYGMH

SEQ ID NO: 6 - protein, 17 aa, Homo sapiens
VISYDGSIKHYADSVKG

SEQ ID NO: 7 - protein, 9 aa, Homo sapiens
DTDYGDIDP

SEQ ID NO: 8 - protein, 11 aa, Homo sapiens
RASQDISSWLA

SEQ ID NO: 9 - protein, 7 aa, Homo sapiens
AASSLQS

SEQ ID NO: 10 - protein, 10 aa, Homo sapiens
QQASSFPSIT